(19) United States
(12) Patent Application Publication
(10) Pub. No.: US 2023/0355870 A1
(43) Pub. Date: Nov. 9, 2023

(54) PRESSURE DEVICE

(71) Applicant: Murata Manufacturing Co., Ltd., Kyoto (JP)

(72) Inventors: Shinichiro MATSUMOTO, Kyoto (JP); Atsushi MATSUMOTO, Kyoto (JP)

(21) Appl. No.: 18/352,617

(22) Filed: Jul. 14, 2023

Related U.S. Application Data
(63) Continuation of application No. PCT/JP2021/044050, filed on Dec. 1, 2021.

(30) Foreign Application Priority Data
Feb. 1, 2021 (JP) 2021-014118

(51) Int. Cl. A61M 5/155; F04B 43/04

(52) U.S. Cl. CPC A61M 5/155; F04B 43/046

(57) ABSTRACT

A pressure device includes a bag-shaped compression bag, a cover member installed at a first surface portion of the compression bag, an accommodating space defined by the cover member and the compression bag, a piezoelectric pump that flows air into the compression bag, and a casing that accommodates the piezoelectric pump. The compression bag has a bag opening formed at a second surface portion different from the first surface portion and connecting an internal space of the compression bag and an outside to each other. The casing has a casing opening. The casing (31) is fixed to the second surface portion of the compression bag while being in contact with the second surface portion to directly connect the bag opening and the casing opening to each other in an overlapping manner.

### CROSS REFERENCE TO RELATED APPLICATION

This is a continuation of International Application No. PCT/JP2021/044050 filed on Dec. 1, 2021 which claims priority from Japanese Patent Application No. 2021-014118 filed on Feb. 1, 2021. The contents of these applications are incorporated herein by reference in their entireties.

### BACKGROUND OF THE DISCLOSURE

### Field of the Disclosure

The present disclosure relates to a pressure device that compresses a bag accommodating a liquid from outside.

### Description of the Related Art

Patent Document 1 describes an intubation feeding device. The device in Patent Document 1 includes an airbag, an electric pump, and an air pressure pipe. The airbag is connected to the electric pump through the air pressure pipe. The airbag includes a bag-shaped compression air chamber and an accommodating space. The compression air chamber and the accommodating space are adjacent to each other, and a nutrition bag is accommodated in the accommodating space.

When the electric pump is driven, air flows into the compression air chamber through the air pressure pipe, and the compression air chamber expands. The expansion of the compression air chamber narrows the accommodating space, and compresses the nutrition bag.
Patent Document 1: Japanese Unexamined Patent Application Publication No. 2011-24817

### BRIEF SUMMARY OF THE DISCLOSURE

However, the device described in Patent Document 1 includes the air pressure pipe between the electric pump and the airbag, and is more likely to reduce the efficiency of air flow into the airbag. In addition, the device is large in size, has limited portability, and presents limitations in terms of the installation site.

Thus, the present disclosure aims to provide a pressure device that has great portability and installability, and improved compression efficiency.

A pressure device according to the present disclosure includes a bag-shaped compression bag, a cover unit installed at a first surface portion of the compression bag, an accommodating space defined by the cover unit and a first surface portion of the compression bag, a piezoelectric pump that flows air into the compression bag, and a casing that accommodates the piezoelectric pump. The compression bag is disposed at a second surface portion different from the first surface portion, and has a bag opening that connects an internal space of the compression bag and an outside to each other. The casing has a casing opening. The casing is fixed to the second surface portion of the compression bag while being in contact with the second surface portion to directly connect the bag opening and the casing opening to each other in an overlapping manner.

In this structure, air flows into the compression bag from the piezoelectric pump without passing through another tubular path. This structure reduces a pressure loss caused by air passing through the tubular path. The piezoelectric pump is directly mounted on the compression bag. Thus, the pressure device has a simpler shape and smaller size.

The present disclosure can provide a pressure device having great portability, installability, and compression efficiency.

### BRIEF DESCRIPTION OF THE SEVERAL VIEWS OF THE DRAWINGS

FIG. 1A is an external perspective view of a pressure device 10 according to a first embodiment, and FIG. 1B is an exploded perspective view of the pressure device 10 according to the first embodiment.

FIG. 2 is an external perspective view of the pressure device 10 to which a liquid accommodating bag BG is mounted.

FIG. 3A is a cross-sectional view of the pressure device 10 according to the first embodiment, and FIG. 3B is an exploded cross-sectional view of the pressure device 10 according to the first embodiment.

FIG. 4A is an enlarged cross-sectional view of a portion where a casing 31 is fixed to a compression bag 21, FIG. 4B is an enlarged cross-sectional view of an example of a fixing method, and FIG. 4C is an enlarged cross-sectional view of a derivative example of a fixed state.

FIG. 5 is a cross-sectional view of a structure example of a piezoelectric pump 32.

FIG. 6A is a cross-sectional view of a pressure device 10A according to a second embodiment, and FIG. 6B is an exploded cross-sectional view of the pressure device 10A according to the second embodiment.

FIG. 7 is an exploded perspective view of a pressure device 10B according to a third embodiment.

FIG. 8A is a cross-sectional view of the pressure device 10B according to the third embodiment, and FIG. 8B is an exploded cross-sectional view of the pressure device 10B according to the third embodiment.

FIG. 9 is an exploded perspective view of a pressure device 10C according to a fourth embodiment.

FIG. 10A is a cross-sectional view of the pressure device 10C according to the fourth embodiment, and FIG. 10B is an exploded cross-sectional view of the pressure device 10C according to the third embodiment.

### DETAILED DESCRIPTION OF THE DISCLOSURE

### First Embodiment

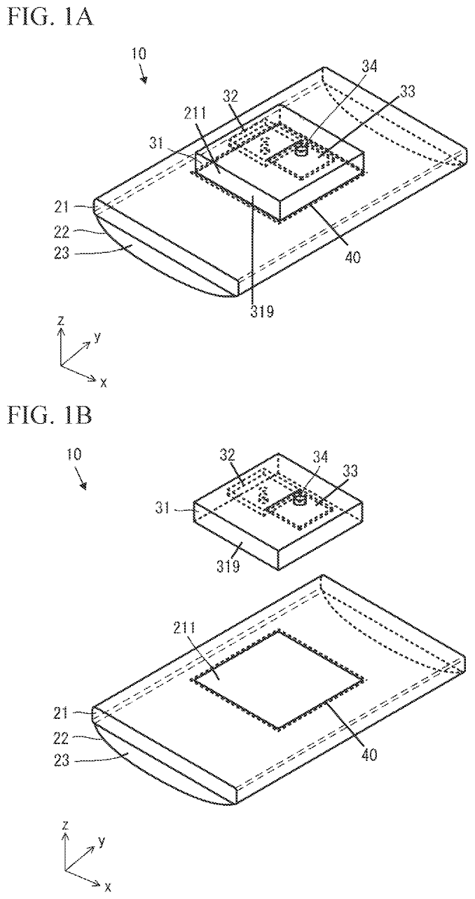

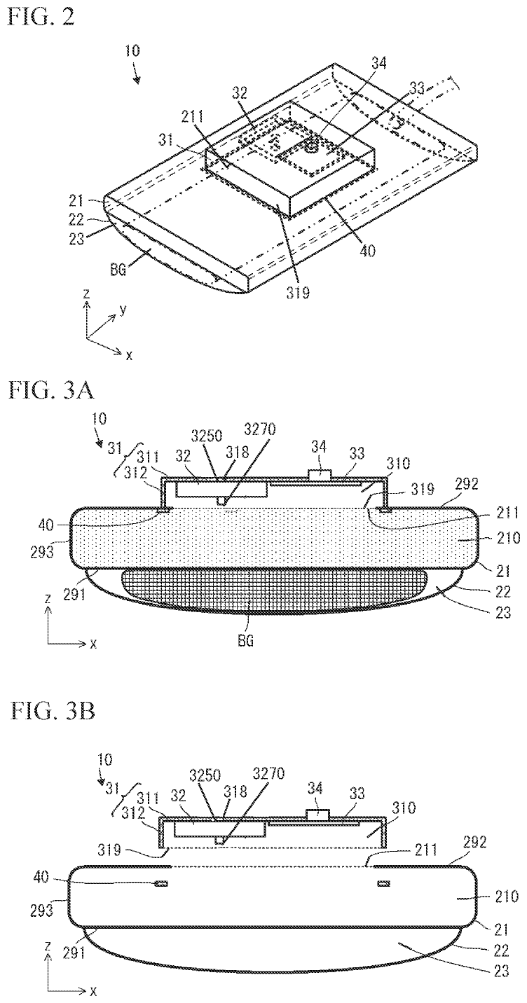

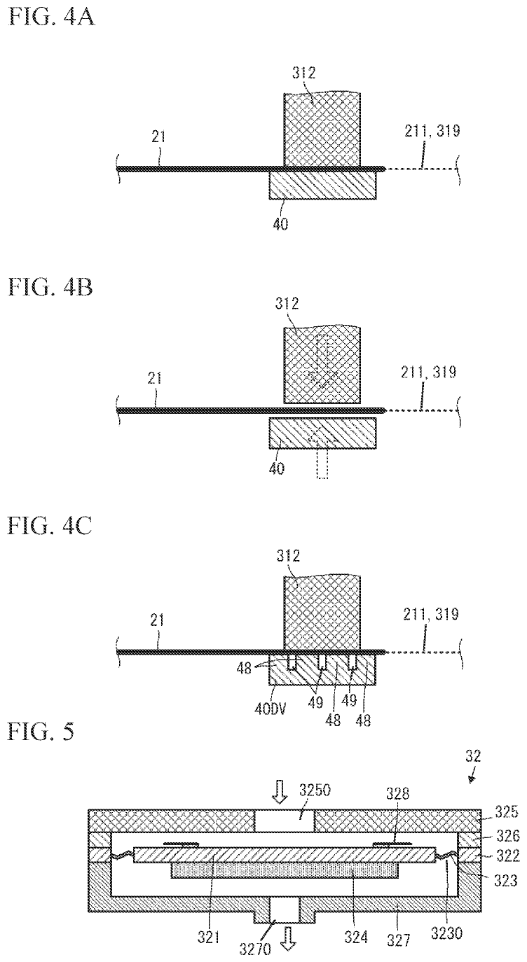

A pressure device according to a first embodiment of the present disclosure is described with reference to the drawings. FIG. 1A is an external perspective view of a pressure device 10 according to a first embodiment, and FIG. 1B is an exploded perspective view of the pressure device 10 according to the first embodiment. FIG. 2 is an external perspective view of the pressure device 10 to which a liquid accommodating bag BG is mounted. FIG. 3A is a cross-sectional view of the pressure device 10 according to the first embodiment, and FIG. 3B is an exploded cross-sectional view of the pressure device 10 according to the first embodiment. FIG. 4A is an enlarged cross-sectional view of a portion where a casing 31 is fixed to a compression bag 21, FIG. 4B is an enlarged cross-sectional view of an example of a fixing method, and FIG. 4C is an enlarged cross-sectional view of a derivative example of a fixed state. For ease of understanding the structure of the pressure device, each of the drawings for embodiments including the present embodiment may illustrate the shapes of the components in a partially or entirely exaggerated manner.

As illustrated in FIG. 1A, FIG. 1B, FIG. 2, FIG. 3A, and FIG. 3B, the pressure device 10 includes the compression bag 21, a cover member 22, the casing 31, a piezoelectric pump 32, a control circuit board 33, a drive switch 34, and a fixing jig 40.

The compression bag 21 is a deformable bag. The compression bag 21 is formed from, for example, a resin film or synthetic fiber such as Tencel, rayon, polyester, or nylon. More specifically, as illustrated in FIG. 3A or FIG. 3B, the compression bag 21 includes a first surface portion 291, a second surface portion 292, and a side surface portion 293. The first surface portion 291 and the second surface portion 292 are planar. Here, the expression “planar” is not limited to the state where the surface is entirely planar, but includes, for example, a state where an entire surface is substantially planar when the piezoelectric pump 32 described below does not allow air to flow into the compression bag 21. In the present embodiment, the state where the entire surface is planar is referred to as a static stationary state.

For example, the first surface portion 291 and the second surface portion 292 are rectangles extending in a first direction (an x direction in FIG. 1A, FIG. 1B, FIG. 2, FIG. 3A, and FIG. 3B) and a second direction (a y direction perpendicular to the first direction and illustrated in FIG. 1A, FIG. 1B, FIG. 2, FIG. 3A, and FIG. 3B). The first surface portion 291 and the second surface portion 292 are not limited to rectangles.

The first surface portion 291 and the second surface portion 292 have substantially the same surface area in the static stationary state. In a plan view in the static stationary state, the first surface portion 291 and the second surface portion 292 overlap one on the other. The plan view is a view viewed in a direction perpendicular to the surfaces of the first surface portion 291 and the second surface portion 292. The first surface portion 291 and the second surface portion 292 are spaced apart in a direction perpendicular to the surfaces of the first surface portion 291 and the second surface portion 292.

The side surface portion 293 is disposed between the first surface portion 291 and the second surface portion 292. The side surface portion 293 is connected to outer edges of the surface of the first surface portion 291 and outer edges of the surface of the second surface portion.

With this structure, the compression bag 21 includes a bag internal space 210 defined by the first surface portion 291, the second surface portion 292, and the side surface portion 293.

The second surface portion 292 of the compression bag 21 has a bag opening 211.

The first surface portion 291 and the second surface portion 292 are preferably formed from an easily deformable material.

The cover member 22 is a deformable sheet. The cover member 22 is formed from a material that is less easily deformable than the first surface portion 291 and the second surface portion 292 of the compression bag 21.

The cover member 22 is disposed at the outer surface of the first surface portion 291 of the compression bag 21. The cover member 22 is connected to the first surface portion 291 at or around both ends of the first surface portion 291 in the x direction. On the other hand, the cover member 22 is not connected to the first surface portion 291 at both ends of the first surface portion 291 in the y direction. Thus, a through-hole that extends through in the y direction is defined by the first surface portion 291 of the compression bag 21 and the cover member 22. This through-hole serves as an accommodating space 23. In other words, the accommodating space 23 is defined by the first surface portion 291 of the compression bag 21 and the cover member 22.

The casing 31 has a box shape. The casing 31 is formed from a material that is less easily deformable than the compression bag 21, such as metal or resin. The casing 31 includes a bottom wall 311 and a side wall 312. The bottom wall 311 is a flat board. The side wall 312 extends along the outer edges of the bottom wall 311 in a direction perpendicular to the plane of the bottom wall 311. The side wall 312 is connected to the outer edges of the bottom wall 311. The space defined by the bottom wall 311 and the side wall 312 serves as a casing internal space 310. The casing internal space 310 is open to the outside of the casing 31 on the side opposite to the bottom wall 311. This opening serves as a casing opening 319. The area of the casing opening 319 is greater than that of the bag opening 211. In this case, for example, the area of the casing opening 319 is substantially equal to the area of the bag opening 211.

The bottom wall 311 of the casing 31 has a suction hole 318.

The piezoelectric pump 32 has a suction hole and an ejection hole. The piezoelectric pump 32 sucks a fluid through the suction hole, and ejects the fluid through the ejection hole. A specific example of the piezoelectric pump 32 is described below. The piezoelectric pump 32 has a low-profile shape, and is accommodated in the casing internal space 310 of the casing 31. At this time, the piezoelectric pump 32 is installed at the bottom wall 311 of the casing 31 to connect the suction hole of the piezoelectric pump 32 to the suction hole 318 of the casing 31.

The control circuit board 33 is a board on which, for example, the drive switch 34 and a circuit element that controls the piezoelectric pump 32 are mounted. The control circuit board 33 is accommodated in the casing internal space 310 of the casing 31, and fixed to the bottom wall 311. At this time, the drive switch 34 is exposed to the outside of the casing 31 through a through-hole formed in the bottom wall 311 of the casing 31.

The fixing jig 40 is a frame. The fixing jig 40 has substantially the same shape as the side wall 312 of the casing 31 when viewed in a direction perpendicular to the opening defined by the frame.

The casing 31 is disposed on the outer surface of the second surface portion 292 of the compression bag 21. At this time, the casing opening 319 and the bag opening 211 are directly connected to each other in an overlapping manner. More specifically, the side wall 312 of the casing 31 is in contact with the outer edges of the bag opening 211 in the second surface portion 292 of the compression bag 21 (more precisely, a looped area similar to the outer edges and surrounding the outer edges) along the outer edges.

As illustrated in FIG. 4A, the fixing jig 40 is located on a surface (inner surface) of the second surface portion 292 of the compression bag 21 opposite to the surface with which the side wall 312 of the casing 31 is in contact. The side wall 312 and the fixing jig 40 hold the second surface portion 292 of the compression bag 21 in between. As illustrated in FIG. 4B, in this case, the side wall 312 and the fixing jig 40 are joined with pressure while holding the second surface portion 292 of the compression bag 21 in between. Thus, the casing 31 is fixed to the compression bag 21 with higher bonding strength.

In this structure, the pressure device 10 compresses the liquid accommodating bag BG in the following manner.

As illustrated in FIG. 2 and FIG. 3A, the liquid accommodating bag BG is accommodated in the accommodating space 23 and held between the first surface portion 291 of the compression bag 21 and the cover member 22. At this time, the piezoelectric pump 32 is not driven.

When the piezoelectric pump 32 is driven, the piezoelectric pump 32 sucks air through the suction hole 318, and ejects the air into the casing internal space 310 of the casing 31. The casing internal space 310 and the bag internal space 210 are directly connected to each other through the casing opening 319 and the bag opening 211. Thus, the air ejected into the casing internal space 310 flows into the bag internal space 210. After this operation is continuously performed, the air accumulates in the space where the bag internal space 210 and the casing internal space 310 are connected to each other. It is hard to deform the casing 31, but easy to deform the compression bag 21. Thus, the volume of the compression bag 21 increases with the amount of the air that flows in. Thus, the compression bag 21 gradually expands. Here, the cover member 22 is less easily deformable than the compression bag 21, and thus, the expansion of the compression bag 21 reduces the volume of the accommodating space 23 between the compression bag 21 and the cover member 22. With the reduction of the volume of the accommodating space 23, the liquid accommodating bag BG is compressed by the first surface portion 291 of the compression bag 21 and the cover member 22 from outside. Thus, the liquid accommodated in the liquid accommodating bag BG is ejected at a predetermined rate.

In this structure, in the pressure device 10, the air ejected by the piezoelectric pump 32 directly flows into the compression bag 21 without passing through a tubular path which is used in the existing technology. This structure reduces a loss of the air flowing into the compression bag 21, and improves the compression efficiency.

The casing 31 including the piezoelectric pump 32 is directly fixed to the compression bag 21. Thus, the pressure device 10 has a simple shape and a small size. Thus, the pressure device 10 can have great portability and installability. Great installability refers to, for example, having great freedom in installation.

In the pressure device 10, the ejection hole of the piezoelectric pump 32 faces the compression bag 21, and no screen is interposed between the ejection hole of the piezoelectric pump 32 and the compression bag 21. Thus, the air ejected from the piezoelectric pump 32 more efficiently flows into the bag internal space 210 in the compression bag 21.

The fixing jig may have a shape illustrated in FIG. 4C. As illustrated in FIG. 4C, a fixing jig 40DV includes multiple grooves 49 extending in a frame shape, and multiple protrusions 48 defined by these grooves 49. The multiple protrusions 48 and grooves 49 alternate in the width direction of the fixing jig 40DV. In this structure, even if either one of the protrusions 48 at both ends in the width direction came off the compression bag 21, the other protrusion 48 would remain bonded to the compression bag 21. This structure can thus prevent the bonded surface of the fixing jig 40DV from entirely coming off the compression bag 21 at a time.

For example, the piezoelectric pump 32 has the following structure. FIG. 5 is a cross-sectional view of an example structure of a piezoelectric pump. A specific structure of the piezoelectric pump 32 is not described in detail, and its schematic structure is described, since the applicants filed patent applications describing various structures in the past.

As illustrated in FIG. 5, the piezoelectric pump 32 includes a vibration plate 321, a frame 322, a support 323, a piezoelectric device 324, a casing flat board 325, a casing side wall member 326, a casing cover member 327, and a valve member 328.

The vibration plate 321 is supported on the frame 322 via the support 323 to vibrate. The piezoelectric device 324 is installed at the vibration plate 321. The frame 322 is connected to the casing flat board 325 with the frame-shaped casing side wall member 326 interposed in between. The frame 322 is connected to the casing cover member 327.

In this structure, the piezoelectric pump 32 has an internal space defined by the frame 322, the casing flat board 325, the casing side wall member 326, and the casing cover member 327. The casing flat board 325 has a suction hole 3250, and the casing cover member 327 has an ejection hole 3270.

When the vibration plate 321 is vibrated by the piezoelectric device 324, the piezoelectric pump 32 sucks air into the internal space from the outside via the suction hole 3250, and ejects air from the internal space to the outside via an ejection hole 3270. At this time, the piezoelectric pump 32 including the valve member 328 transports air from the suction hole 3250 to the ejection hole 3270, and restricts backflow of air from the ejection hole 3270 to the suction hole 3250. Herein, a structure where the valve member 328 is installed at the vibration plate 321, that is, a structure where the valve member 328 is installed in the internal space in the piezoelectric pump 32 is described. Instead of the valve member 328, a valve device may be disposed on the outer side of the suction hole 3250 in the piezoelectric pump 32 or on the outer side of the ejection hole 3270.

With this structure, the piezoelectric pump 32 can have a low profile. Thus, the casing 31 can have a low profile, and the pressure device 10 can have a smaller size. Particularly, as the valve member 328 is disposed in the piezoelectric pump 32, the casing 31 can have a lower profile, and the pressure device 10 can have a smaller size.

### Second Embodiment

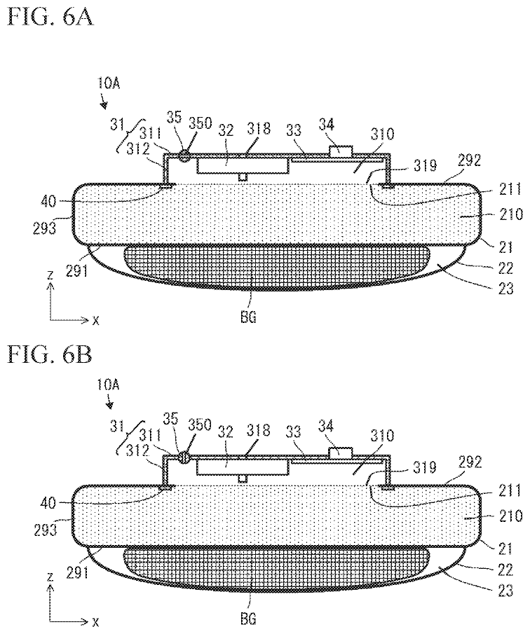

A pressure device according to a second embodiment of the present disclosure is described with reference to the drawings. FIG. 6A is a cross-sectional view of a pressure device 10A according to a second embodiment, and FIG. 6B is an exploded cross-sectional view of the pressure device 10A according to the second embodiment.

As illustrated in FIG. 6A and FIG. 6B, the pressure device 10A according to the second embodiment differs from the pressure device 10 according to the first embodiment in that it includes a valve 35. Other components in the pressure device 10A are the same as those in the pressure device 10, and thus are not described.

The valve 35 is installed at the casing 31. For example, as illustrated in FIG. 6A and FIG. 6B, the valve 35 is installed at the bottom wall 311 of the casing 31. The valve 35 switches between opening and shutting the casing internal space 310 of the casing 31 with respect to the external space. For example, in FIG. 6A, the valve 35 shuts the connection between the casing internal space 310 and the external space. In FIG. 6B, the valve 35 connects the casing internal space 310 and the external space to each other.

In this structure, the valve 35 shuts the connection between the casing internal space 310 and the external space during the compression. The valve 35 connects the casing internal space 310 and the external space to each other during the decompression.

In this structure, the pressure device 10A can efficiently flow air into the compression bag 21 during the compression. The pressure device 10A can efficiently discharge air in the compression bag 21 to the outside during the decompression such as after use.

### Third Embodiment

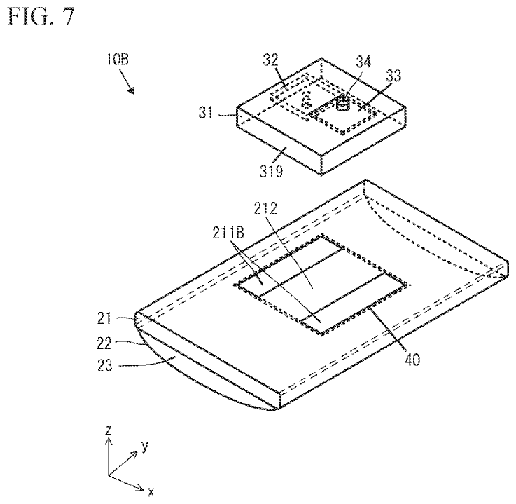

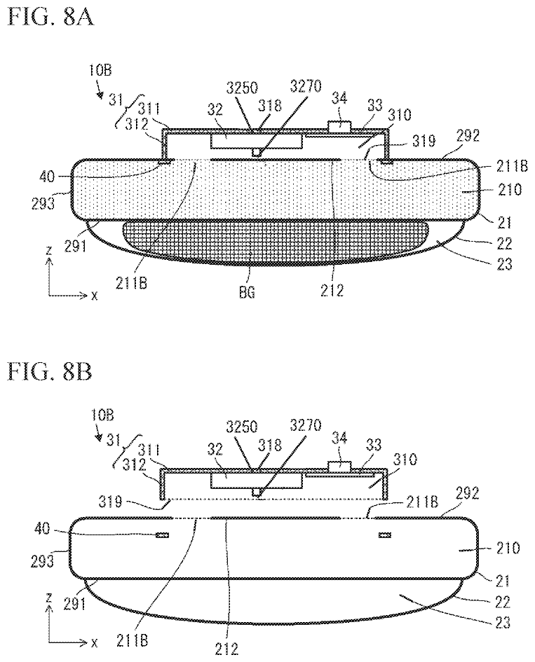

A pressure device according to a third embodiment of the present disclosure is described with reference to the drawings. FIG. 7 is an exploded perspective view of a pressure device 10B according to a third embodiment. FIG. 8A is a cross-sectional view of the pressure device 10B according to the third embodiment, and FIG. 8B is an exploded cross-sectional view of the pressure device 10B according to the third embodiment.

As illustrated in FIG. 7, FIG. 8A, and FIG. 8B, the pressure device 10B according to the third embodiment differs from the pressure device 10 according to the first embodiment in that it includes a bag opening 211B. Other components of the pressure device 10B are the same as those in the pressure device 10, and thus are not described.

The compression bag 21 has the bag opening 211B. A screen 212 is disposed at the bag opening 211B to partially close the bag opening 211B. The screen 212 has a film shape. For example, the screen 212 extends in the y direction, and is connected to the second surface portion 292 of the compression bag 21 at both ends in the y direction.

With this structure, the bag opening 211B is divided in the x direction into two openings on both sides of the screen 212. Thus, the air ejected from the piezoelectric pump 32 is divided into two branches in the x direction, which then flow into the bag internal space 210 in the compression bag 21. Thus, the difference in the air flow rate between positions in the compression bag 21 in the x direction can be reduced. Specifically, the pressure device 10B can more uniformly flow air into the compression bag 21.

At this time, as illustrated in FIG. 8A and FIG. 8B, when the surface of the second surface portion 292 is viewed in a plan, the ejection hole 3270 of the piezoelectric pump 32 preferably overlaps the screen 212. Thus, the air ejected from the piezoelectric pump 32 hits against the screen 212, and then flows into the two openings of the bag opening 211B. Thus, the pressure device 10A can more uniformly flows air into the compression bag 21.

Although the pressure device 10B includes one screen 212, the number of the screen 212 is not limited to this, and the screen 212 may have another shape.

### Fourth Embodiment

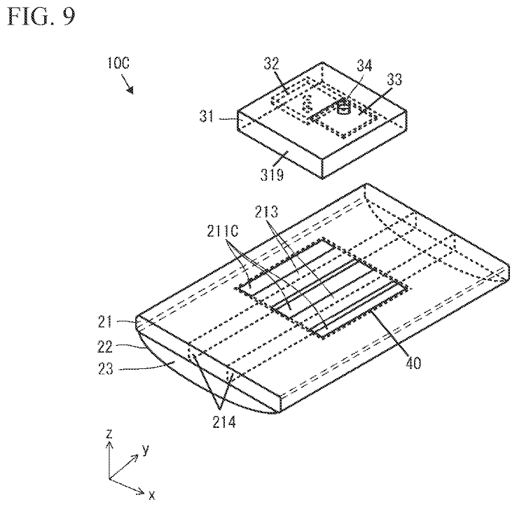

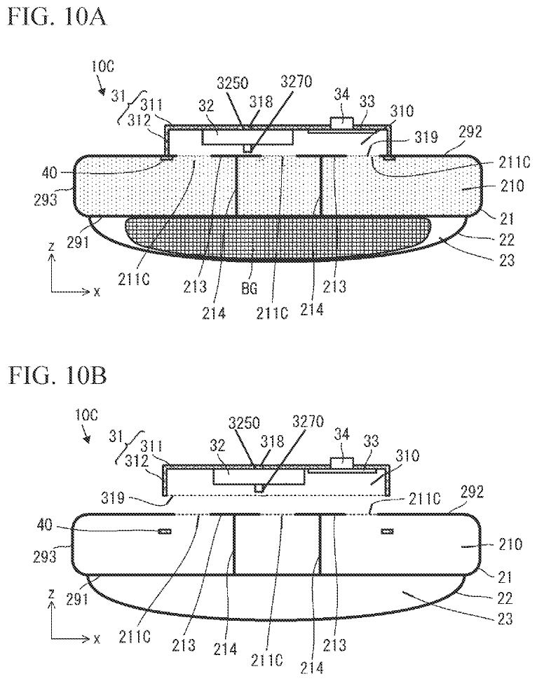

A pressure device according to a fourth embodiment of the present disclosure is described with reference to the drawings. FIG. 9 is an exploded perspective view of a pressure device 10C according to a fourth embodiment. FIG. 10A is a cross-sectional view of the pressure device 10C according to the fourth embodiment, and FIG. 10B is an exploded cross-sectional view of the pressure device 10C according to the third embodiment.

As illustrated in FIG. 9, FIG. 10A, and FIG. 10B, the pressure device 10C according to the fourth embodiment differs from the pressure device 10B according to the third embodiment in that it includes bag openings 211C and multiple partitioning members 214. Other components of the pressure device 10C are the same as those in the pressure device 10B, and thus are not described.

The compression bag 21 includes bag openings 211C. Two screens 213 are disposed at the bag openings 211C to partially close the bag openings 211C. The screens 213 have a film shape. For example, the screens 213 extend in the y direction, and are connected to the second surface portion 292 of the compression bag 21 at both ends in the y direction. In this structure, the bag openings 211C and the screens 213 alternate in the x direction.

The compression bag 21 includes two partitioning members 214. The partitioning members 214 extend in the y direction to partition the bag internal space 210 into three chambers in the x direction. The two partitioning members 214 are located to overlap the two screens 213.

In this structure, the air ejected from the piezoelectric pump 32 is divided into three branches in the x direction. The branches of the air flow into the three chambers partitioned by the partitioning members 214 in the bag internal space 210 in the compression bag 21. Thus, the difference in the air flow rate at positions in the compression bag 21 in the x direction can be reduced. Specifically, the pressure device 10C can more uniformly flow air into the compression bag 21.

The third embodiment and the fourth embodiment illustrate a structure where one or more screens are disposed at the compression bag 21. However, one or more screens may be disposed at the casing 31.

Components between the different embodiments may be combined with each other as appropriate. Each combination can exert its own operation effects.
10, 10A, 10B, 10C pressure device
21 compression bag
22 cover member
23 accommodating space
31 casing
32 piezoelectric pump
33 control circuit board
34 drive switch
35 valve
40 fixing jig
40DV fixing jig
48 protrusion
49 groove
210 bag internal space
211, 211B, 211C bag opening
212, 213 screen
214 partitioning member
291 first surface portion
292 second surface portion
293 side surface portion
310 casing internal space
311 bottom wall
312 side wall
318 suction hole
319 casing opening
321 vibration plate
322 frame
323 support
324 piezoelectric device
325 casing flat board
326 casing side wall member
327 casing cover member
328 valve member
3250 suction hole
3270 ejection hole
BG liquid accommodating bag

1. A pressure device, comprising:
a bag-shaped compression bag;
a cover member installed at a first surface portion of the compression bag;
an accommodating space defined by the cover member and the first surface portion of the compression bag;
a piezoelectric pump configured to allow air to flow into the compression bag; and
a casing accommodating the piezoelectric pump,
wherein the compression bag has a bag opening connecting an internal space of the compression bag and an outside to each other,
wherein the bag opening is disposed at a second surface portion different from the first surface portion,
wherein the casing has a casing opening, and
wherein the casing is fixed to the second surface portion of the compression bag while being in contact with the second surface portion to directly connect the bag opening and the casing opening to each other in an overlapping manner.

2. The pressure device according to claim 1, further comprising:
a fixing jig disposed in the internal space of the compression bag to hold the compression bag between the casing and the fixing jig.

3. The pressure device according to claim 1, wherein an area of the bag opening is equal to an area of the casing opening.

4. The pressure device according to claim 1, wherein the casing includes a valve configured to switch between opening and shutting an internal space of the casing with respect to an external space.

5. The pressure device according to claim 1, further comprising a screen disposed at the bag opening or the casing opening to partially close the bag opening or the casing opening.

6. The pressure device according to claim 5, wherein, when viewed in a direction perpendicular to the second surface portion, an ejection hole of the piezoelectric pump overlaps the screen.

7. The pressure device according to claim 1, wherein the compression bag includes a partitioning member partitioning the internal space of the compression bag into a plurality of chambers when viewed in a direction perpendicular to the second surface portion.

8. The pressure device according to claim 2, wherein an area of the bag opening is equal to an area of the casing opening.

9. The pressure device according to claim 2, wherein the casing includes a valve configured to switch between opening and shutting an internal space of the casing with respect to an external space.

10. The pressure device according to claim 3, wherein the casing includes a valve configured to switch between opening and shutting an internal space of the casing with respect to an external space.

11. The pressure device according to claim 2, further comprising a screen disposed at the bag opening or the casing opening to partially close the bag opening or the casing opening.

12. The pressure device according to claim 3, further comprising a screen disposed at the bag opening or the casing opening to partially close the bag opening or the casing opening.

13. The pressure device according to claim 4, further comprising a screen disposed at the bag opening or the casing opening to partially close the bag opening or the casing opening.

14. The pressure device according to claim 2, wherein the compression bag includes a partitioning member partitioning the internal space of the compression bag into a plurality of chambers when viewed in a direction perpendicular to the second surface portion.

15. The pressure device according to claim 3, wherein the compression bag includes a partitioning member partitioning the internal space of the compression bag into a plurality of chambers when viewed in a direction perpendicular to the second surface portion.

16. The pressure device according to claim 4, wherein the compression bag includes a partitioning member partitioning the internal space of the compression bag into a plurality of chambers when viewed in a direction perpendicular to the second surface portion.

17. The pressure device according to claim 5, wherein the compression bag includes a partitioning member partitioning the internal space of the compression bag into a plurality of chambers when viewed in a direction perpendicular to the second surface portion.

18. The pressure device according to claim 6, wherein the compression bag includes a partitioning member partitioning the internal space of the compression bag into a plurality of chambers when viewed in a direction perpendicular to the second surface portion.
